(19) United States
(12) Patent Application Publication
(10) Pub. No.: US 2024/0315733 A1
(43) Pub. Date: Sep. 26, 2024

(54) System and Method for Performing Distraction Osteogenesis

(71) Applicant: Nelson Mandela University, Summerstrand, Port Elizabeth (ZA)

(72) Inventors: Shahrokh Hatefi, Summerstrand, Port Elizabeth (ZA); Farouk Smith, Summerstrand, Port Elizabeth (ZA); Khaled Abou-el-Hossein, Summerstrand, Port Elizabeth (ZA)

(21) Appl. No.: 18/578,145

(22) PCT Filed: Jul. 15, 2022
(86) PCT No.: PCT/ZA2022/050032

§ 371 Date: Jan. 10, 2024

(30) Foreign Application Priority Data
Jul. 16, 2021 (ZA) 2021/05003

(51) Int. Cl. A61B 17/66; A61B 17/00

(52) U.S. Cl. CPC A61B 17/66; A61B 2017/00141

(57) ABSTRACT

The invention provides a system for performing distraction osteogenesis of a hand or other anatomical extremity. The system includes a proximal bone fixture component configured to engage a proximal portion of a bone segment, and a movable distractor component which can be positioned distal of the proximal bone fixture component and which is configured to engage a region of the hand located distal of the bone portion engaged by the bone fixture component. Drive means are provided for applying a distraction force between the bone fixture component and the distractor component, and a control component is provided to control operation of the drive means automatically. The drive means can be operated in a continuous manner. A user interface module is provided and can be separated from the control component after the system's operating parameters have been set. The invention also provides a method of performing distraction osteogenesis automatically.

### CROSS-REFERENCE TO RELATED APPLICATION

This application claims priority from South African provisional patent application number 2021/05003 filed on 16 Jul. 2021, which is incorporated by reference herein.

### FIELD OF THE INVENTION

This invention relates to distraction osteogenesis. A system and method are provided which may be used in the lengthening of bones affected by acquired or congenital bone defects.

### BACKGROUND TO THE INVENTION

Distraction osteogenesis (DO), also called distraction, callotasis and osteodistraction, is a technique used to reconstruct bone tissue along with the surrounding soft tissue, instead of performing conventional surgical techniques (including bone grafting), or to extend congenitally short bones or repair skeletal deformities in various body zones. The DO technique provides a way to make a longer bone out of a shorter one. After a bone is cut during surgery, a device termed a distractor may be used to pull the two pieces of bone apart slowly. New bone grows (osteogenesis) to fill the gap.

In the DO process, a bone segment (BS) may be osteotomized from healthy bone tissue near the defected zone in which the BS is still connected to the surrounding soft tissue. In the next step, the distractor is installed on the bone and fixed to the main bone tissue via a constant or stationary component of the distractor while a movable component is connected to the osteotomized moving section or portion of the BS using fixed screws. The distractor is configured to move the BS along a linear vector or trajectory towards a destination position with controlled distraction parameters (including rate and rhythm of distraction). After the installation of the distractor, there is typically a latency phase of three to seven days during which the distractor remains inactive and bone tissue regeneration occurs between the main bone part and the osteotomized BS. Subsequently, the activation phase begins, and the BS is moved towards the destination position by applying a distraction force.

The field of DO has received more attention than conventional bone reconstruction methods, including bone grafting, prosthetics, and the vascularized flap. This is due to a higher quality of reconstructed bone tissue, shorter time of the reconstruction process, and fewer complications during and after the treatment.

A recent development in the DO field is Automatic Continuous Distraction Osteogenesis (ACDO) which provides an automatically controlled and executed process for making continuous movements of the BS under reconstruction. To date, ACDO devices and techniques have been limited in their application to the reconstruction of bones in the oral and maxillofacial regions.

These devices cannot be used for reconstruction of bones in other body zones as treatment conditions and DO protocols are different. Specific systems and methods need to be used in reconstruction of bones in different body zones.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with an aspect of the invention there is provided a system for performing distraction osteogenesis of a bone segment of an anatomical extremity of a human or animal subject, said system comprising:
a proximal bone fixture component configured to engage a proximal portion of said bone segment;
a movable distractor component configured to be positionable distal of the proximal bone fixture component, and to engage a region of said anatomical extremity located distal of the bone portion engageable by the bone fixture component;
drive means configured to apply a distraction or separating force between the bone fixture component and the movable distractor component; and
a control component arranged to control operation of said drive means automatically.

The anatomical extremity may comprise a hand.

The control component may be configured to operate the drive means in a continuous manner.

The control component may include a user interface module, also referred to in this specification as a human-machine interface (HMI), for setting parameters of automatic operation and control of the drive means. The user interface module may be configured to be removable or separable from the remainder of the control component and system, optionally in a tool-free manner. The term “automatic distractor device” is used in this specification to refer to the part of the system that remains after the user interface module has been separated from it.

The proximal bone fixture component may be configured as a constant bone fixture component so that, in use, it provides a constant fixture to the bone segment during the osteogenesis. For the duration of the osteogenesis procedure, therefore, the proximal bone fixture component may be maintained in a substantially stationary or immobilised position relatively to the remainder of the hand or other anatomical extremity undergoing osteogenesis.

The proximal bone fixture component may include proximal bone fixture means such as, but not limited to, one or more fixed screws or other suitable fasteners.

The proximal bone fixture component and the movable distractor component may interconnect via a transitional arrangement comprising force transfer means configured to apply the distraction or separating force between the components. The system may include a support structure such as a body, housing, or frame for mounting and supporting the transitional arrangement and the components.

The drive means may be configured as a linear drive means. The linear drive means may be connected to the movable distractor component and may be configured to move the distractor component along a linear vector or trajectory away from the bone fixture component. The drive means may be configured to pull or push the former away from the latter, thereby to apply the distraction or separating force between the two components.

The drive means may be configured to apply a continuous distraction force to move the BS in a fully automatic DO process.

The movable distractor component may comprise grip means for gripping the distal region of the hand or other anatomical extremity, e.g., means for gripping a distal segment or phalange of a finger being lengthened, or a bone fragment in that region. The grip means may accordingly comprise distal bone fixture means such as, but not limited to, one or more fixed screws. Instead, or in addition, the grip means may comprise other suitable means capable of engaging with the distal region of the hand or other anatomical extremity.

The system may include guide means arranged to provide guided linear movement of the movable distractor component relatively to the bone fixture component.

In accordance with a further aspect of the invention there is provided a method for performing distraction osteogenesis of a bone segment of an anatomical extremity of a human or animal subject, comprising the steps of
fixing a proximal bone fixture component to a proximal portion of the bone segment;
utilising a movable distractor component to engage a region of said anatomical extremity positioned distal of said proximal bone portion;
utilising a drive means to apply a distraction or separating force between the bone fixture component and the movable distractor component; and
controlling operation of said drive means automatically using a control component.

The anatomical extremity may comprise a hand.

Further details of the fixture component, distractor component, drive means, and control component may be as hereinbefore described.

The method may include operating the control component to drive the drive means in a continuous manner.

The control component may include a user interface module for setting parameters of automatic operation and control of the drive means, and the method may include setting such parameters with the user interface module. The user interface module may be configured to be separable from the control component in a tool-free manner and the method may include separating the module from the remainder of the control component after the parameters have been set.

The method may include utilizing the drive means to move the distractor component along a linear vector away from the proximal bone fixture component.

Embodiments and modes of performing the invention will now be described, by way of example only, with reference to the accompanying drawings.

### BRIEF DESCRIPTION OF THE DRAWINGS

In the drawings:

FIG. 1 is a schematic block diagram of an embodiment of a system for performing automatic continuous distraction osteogenesis of a bone segment of a hand;

FIG. 2 is a schematic model diagram of the embodiment of FIG. 1;

FIG. 3 is a schematic three-dimensional, dorsolateral view of an automatic distractor device forming part of the embodiment of the system shown in FIG. 2;

FIG. 4 is a schematic proximal dorsal view of an automatic distractor device shown engaged with the right forefinger of a patient undergoing a distraction osteogenesis procedure for hand bone reconstruction;

FIG. 5 is a first schematic dorsal view of an automatic distractor device shown engaged with a patient's middle finger in a starting condition prior to activation of the device to perform a distraction osteogenesis procedure;

FIG. 6 is a second schematic dorsal view of the automatic distractor device shown in FIG. 5, with the device in this instance being illustrated during operation as it moves towards a destination position, thereby generating bone tissue in the patient's middle finger by automatic distraction osteogenesis; and

FIG. 7 is a schematic controller circuit diagram which illustrates an example of a control component for the disclosed system.

### DETAILED DESCRIPTION WITH REFERENCE TO THE DRAWINGS

Embodiments of the disclosed system are explained in greater detail in the following description. Aspects of the present disclosure provide a system for performing automatic continuous distraction osteogenesis (ACDO) on anatomical extremities such as, but not limited to, hands.

The system may include a stationary or constant bone fixture component and a movable distractor component arranged to engage with proximal and distal regions of a hand, respectively. These two components may be arranged to oppose each other and may be interconnected via a transitional arrangement and force transfer means, so that the movable distractor component can be gradually and continuously driven away from the bone fixture component by applying a distraction or separating force between the two components. This may then permit a distal, osteotomized fragment of the targeted BS to be pulled or pushed away from the main or distal portion thereof, which in turn may induce osteogenesis of bone tissue to occur between the distal fragment and the main portion of the BS. As a result, the BS may be gradually lengthened or elongated as the distal fragment is moved slowly away from the main portion.

The bone fixture component may be configured to be fixable to the proximal portion or section of the BS. The bone fixture component may be configured so that it provides a constant, stationary or immobilised fixture relatively to the remainder of the hand. The bone fixture component may include a bone fixture with at least one screw or other fastener for engaging with the BS. The bone fixture component may be shaped, dimensioned and configured to be fixable to a hand bone. For example, the fixture component may be shaped, dimensioned and configured to fixable to at least one hand bone selected from the group consisting of carpal bones, metacarpal bones, and proximal, intermediate and distal phalanges of fingers and thumbs. The system may accordingly be suitable for lengthening, reconstructing, correcting or generating hand bones of these types. The system may facilitate treatment of acquired and congenital shortening of hand bones. The system may also be applied for lengthening grafted finger bones or toe bones.

The movable distractor component may be configured to be positionable distal of the proximal bone fixture component and to engage a distal region of the hand, i.e., a region of the hand that is located distal of the proximal bone portion to which the stationary bone fixture component is fixed. In use, the distractor component may be engaged with the distal region by fixing it to a bone fragment in that region, or by other means of engagement discussed further below. If the distractor component is equipped with a bone fixture, this may include at least one screw or other fastener like those employed for fixing the constant bone fixture component to the bone segment. In use, linear movement of the distractor component away from the bone fixture component may gradually pull the distal bone fragment away from the main portion of the BS, thereby inducing osteogenesis to occur between the bone fragment and the main portion of the BS.

The disclosed system typically includes a drive means and control component. The drive means may be configured to apply the distraction force between the bone fixture component and the movable distractor component, while the control component may be arranged to control operation of the drive means automatically during performance of the distraction osteogenesis.

To perform continuous distraction osteogenesis, a linear continuous force should be applied to the BS to continuously move it towards a desired destination position along a linear path, vector or trajectory. Accordingly, the drive means may be configured to apply a linear distraction force.

The control component may be configured to operate the drive means automatically.

The disclosed DO system may be configured to operate the drive means in a continuous manner, i.e., to provide continuous distraction motion. The disclosed system may accordingly be provided as an Automatic Continuous Distraction Osteogenesis (ACDO) system, device, or apparatus. For purposes of the present specification, the term “continuous” and variations thereof such as “continuously” will be understood to have their widest meaning. For example, in the present context the terms will be understood to refer not only to a constant and unbroken distraction motion, but also to a set of intermittent or stepwise distraction movements delivered automatically over the course of a distraction procedure. Such movements may be applied in a regulated and gradual manner. Accordingly, although the term “continuous” and its variations may typically connote a constant or ongoing distraction motion, for present purposes they may equally connote a process involving multiple intermittent or periodic phases of distraction motion performed over a period. By way of non-limiting example, such motion may be produced using a stepper motor which works in a series of discrete steps instead of one constant motion. The continuous motion of the disclosed system may accordingly be provided as a controlled set of intermittent steps or phases triggered and controlled automatically by the control component. The automated steps may together establish a generally regular rhythm of distraction movements over the period.

The control component may include a user interface module or HMI for setting parameters and working factors required for the subsequent automatic operation and control of the drive means. The HMI may include output means, such as one or more displays. It may further include input means such as a keypad, touchpad, or touch-sensitive display. The input and output means may be coupled to or integrally formed with the user interface module. The display may be coupled via a display adaptor.

The HMI may be configured to be removable or separable from the automatic distractor device once the operational parameters have been set. Typically, although not necessarily, the HMI is configured to be separable from the device in a tool-free manner, that is, without the need for any tools to be used to effect the separation.

The drive means may be configured as a linear drive means or assembly. The linear drive means may be connected to the distractor component and may be configured to move the distractor component along the required linear vector or trajectory away from the bone fixture component. It may optionally also be configured to move the former towards the latter when, for example, the linear actuator and distractor component must be reset and returned from their destination positions to their starting positions.

The linear drive means may be configured to apply the continuous distraction force in a fully automated DO process controlled by the control component.

It will be appreciated that numerous mechanisms and systems are available for implementing the linear drive means. The linear drive means may accordingly be operable by any suitable mechanism, including but not limited to mechatronic, electromechanical, hydraulic, or pneumatic mechanisms. Amongst other suitable types of drive means, a lead screw, worm drive, or piston arrangement may be suitable. The linear drive means may thus comprise a motor cooperating with a cantilevered lead screw translation mechanism configured to pull or push the distractor component away from the bone fixture component.

The movable distractor component may comprise grip means for gripping the distal region of the hand or other anatomical extremity, e.g., a segment of a finger being lengthened. The distractor component may accordingly be configured to grip or fix to bone, skin, or tissue of the distal region.

The grip means may include any suitable apparatus or materials for gripping the distal region of the hand which will be pulled or pushed away from the BS. The grip means may, for example, include screws or other BS fixing formations, or clamping means, or frictional or adhesive means, or any other suitable gripping apparatus or materials. The BS fixing formations may be configured to engage with an osteotomized fragment of the BS. The configuration or properties of the grip means must be adequate to overcome forces opposing the distraction force being applied by the drive means during use of the system, so that the distal portion of the BS can be driven away from the proximal portion of the BS fixed to the constant or stationary bone fixture component.

In one embodiment of the system, the distractor component may comprise a partial or complete ring formation that is shaped, dimensioned, and configured to embrace and grip a distal portion of the anatomical extremity, e.g., the finger being lengthened.

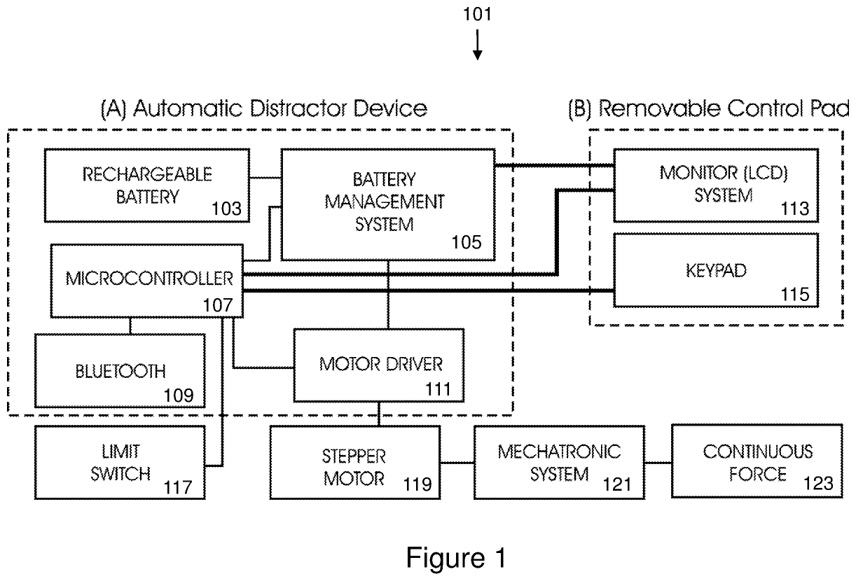

Referring now to the drawings, FIG. 1 is a schematic block diagram illustrating an embodiment (101) of the disclosed system. The system (101) may comprise two main sub-assemblies, namely an automatic distractor device or module (A) and a user interface module or HMI comprising a removable control pad (B).

The automatic distractor device (A) may comprise a rechargeable battery pack (103) and battery management system (105) for managing power supply, a control component comprising a microcontroller (107), a Bluetooth® module (109), and a motor driver or motor control unit (111). In use while powered by the battery (103), the microcontroller (107) may exercise control authority over the motor driver (111). Together, these components may be configured to generate an accurate, reliable, and stable continuous distraction force.

The microcontroller (107) may include a processor for executing computer-readable program code to control the functions of the various components of the system. The control may be exercised by software units executing on the processor, or by hardware. The software units may be configured to be executable by a processing circuit and may be stored in a memory component, and instructions may be provided to the processor to carry out the functionality of the system components. In some cases, for example in a cloud computing implementation, software units arranged to manage and/or process data on behalf of the processor may be provided remotely. Some or all the software units may be provided by a software application downloadable onto and executable on the processor or a remote device.

The removable control pad (B) may comprise a display or monitor system (113) and a keypad (115).

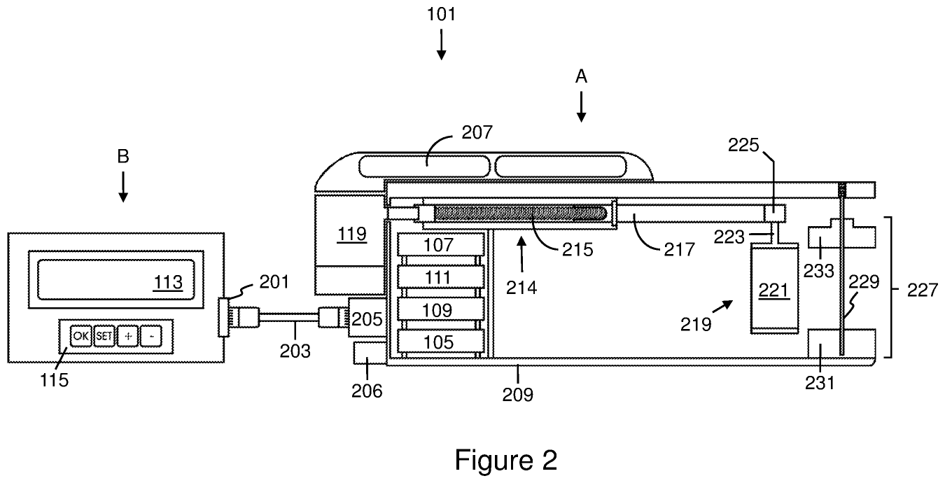

The system (101) may further comprise a limit switch (117) in communication with the microcontroller (107), a stepper motor (119) controlled by the motor driver or motor control unit (111); and a mechatronic system (121) configured to deliver a continuous distraction force (123) between proximal and distal portions of a BS. The mechatronic system (121) is described in greater detail in relation to FIG. 2, below.

Different types of bipolar and unipolar stepper motors can be connected to the control system. In certain non-limiting embodiments of the disclosed system, the control system is capable of driving 5-24 VDC hybrid, bipolar, and unipolar stepper motors.

The control system may be configured to manage the entire distraction process, communicate with the operator, and drive the mechatronic system (121) to generate a continuous distraction force to pull or push the distal portion of the BS away from the proximal portion.

The removable control pad (B) may be used to set and monitor working parameters of the distractor device (A) before and during the bone reconstruction process.

The microcontroller (107) may be connected to the Bluetooth® module (109). The Bluetooth® module (109) may enable instructions and data to be communicated wirelessly between the microcontroller (107) and a remote mobile device or the removable control pad (A) after it has been disconnected. The Bluetooth® module (109) may, for example, be used for wirelessly setting and modifying the working and process parameters of the system (101) during the DO procedure, by execution of suitable software or an application running on a remote device such as a mobile device or the removable control pad (B), or locally on the microcontroller (107). By way of example only, the Bluetooth® module (109) may comprise a serial Bluetooth module such as, but not limited to, a serial HM-10 CC2540 Bluetooth®4.0 unit.

The microcontroller (107) may further be connected to the motor driver (111) and may cooperate with it to drive the stepper motor (119) and thereby operate the mechatronic system (121) in a continuous manner, either constantly or gradually in discrete steps over a period.

The limit switch (117) may be used to set a start position under control of the microcontroller (107).

FIG. 2 is a schematic model diagram of the embodiment (101) of the disclosed system.

Referring to the figure, the system (101) may comprise the following components:
the removable control pad (A) comprising the keypad (115), the display (113), and a CAT6 UTP (Unshielded Twisted Pair) socket (201);
the automatic distractor device (B) comprising:
   a CAT6 UTP network cable (202) and socket (205);
   a power jack (206);
   the stepper motor (119);
   the removable battery pack (103) comprising Lithium-ion batteries or cells (207);
   a support structure or body (209) for holding the various system components;
   a linear drive assembly connected to the stepper motor (119), comprising a solid drive shaft (211), a shaft coupling (213) and a lead screw translation mechanism (214) which includes a threaded lead screw (215) engaged with a complementarily-threaded, tubular linear actuator (217);
   a control module or housing (218) containing the microcontroller (107), the motor driver (111), the Bluetooth® module (109), and the power management system or module (105);
   a movable distal distractor component (219) comprising a titanium ring formation (221) connected via an adjustable linkage (223) to a solid fixture (225), i.e., an end cap or plug on the linear actuator (217); and
   a bone fixture component (227) configured to be fixable to a proximal portion or section of a BS in the hand. The bone fixture component (227) may include at least one titanium fixing screw (229). The screw (229) may have a drill at its point and a screw thread at its rear or upper end. This may be used to fix the automatic distractor device (A) onto a patient's hand by connecting the bone fixture component (227) to the fixed bone section. The screw (229) may be held between a base element or mechanical fixture (231) and a movable element or mechanical fixture (233). The latter fixture (233) may be adjusted to a required height above the base element (231), so that it may press down on the bone section or on the outside of a finger or other hand portion enclosing the fixed bone section.

The mechatronic system (121) in the block diagram of FIG. 1 may include the combination of the linear drive assembly described above, as well as the movable distractor component (219) and the constant bone fixture component (227).

In one exemplary embodiment, the lead screw (215) may have a 1 mm lead, a 1 mm pitch, and a length of 40 mm. The solid drive shaft (211) may have a length of about 4 mm and may connect the lead screw (215) to the shaft (not shown) of the stepper motor (119).

In use, the linear drive assembly may act to translate the controlled and continuous rotation of the stepper motor's shaft into a continuous and smooth linear motion with low ripple and noise, thereby pulling the BS with a continuous force.

The control component may be capable of generating control signals to drive the stepper motor in a micro-stepping drive mode (1/32) via a gearbox with a translation ratio of 1/64. Based upon these specifications and others relating to the control component, stepper motor, and linear mechanism, it may be expected that the illustrated embodiments of the disclosed system may be capable of delivering controlled linear motion with a maximum accuracy of about 7.5 nanometers, while generating a continuous distraction force to pull the BS.

As mentioned, the body (209) of the automatic distractor device (B) may comprise a housing or frame for holding and supporting the system components. The bone fixture component (227) and the movable distractor component (219) may be mounted in the body (209), such that the two components may operate in opposition to each other and are able to counteract each other during the application of the distraction force between them.

In certain embodiments of the system (not shown), guide means may be provided, configured to guide linear movement of the distractor component relatively to the constant bone fixture component. The guide means may, for example, comprise at least one guide rail or rod engageable with a complementary slider or carriage. The guide means may, instead or in addition, comprise any suitable cooperating guide formations, such as rail and groove formations or other arrangements. The guide means are an optional feature since the linear drive means itself may be configured to act as its own guide means (as illustrated in the drawings).

After the installation of the automatic distractor device (A) on the hand bone, the removable control pad (B) may be used to set the input parameters and working factors of the device, including the length of distraction and the distraction rate. In use, the microcontroller (107) may analyse the input data and calculate the required distraction parameters based on the input data. When the activation phase of the DO treatment is commenced, the system (101) starts working and generates a continuous pulling force to move the BS towards the set destination position along the required linear vector. The control component provides precise regulation of the stepper motor (119) and controls the position of motor's drive shaft (211).

In use of the system, by rotating the lead screw (215), the linear actuator (217) may be moved along the required linear vector, thereby moving the titanium ring formation (221) which in turn distracts the distal portion of the BS away from the fixed proximal portion.

The mechanical fixtures (231, 233) of the constant bone fixture component (227), i.e., the base and movable elements, may optionally be provided with a resiliently compressible cushioning material to enhance the fit of the fixtures onto the fixed bone section or around a part of a finger enclosing the fixed bone section.

The power jack (206) may be used to interface the system with an external power supply. The removable battery pack (103) is provided so that the automatic distractor device (A) may be portable. The power management system or module (105) is used to manage the battery pack (103) so that it can run the system. It also controls charging of the battery pack (103).

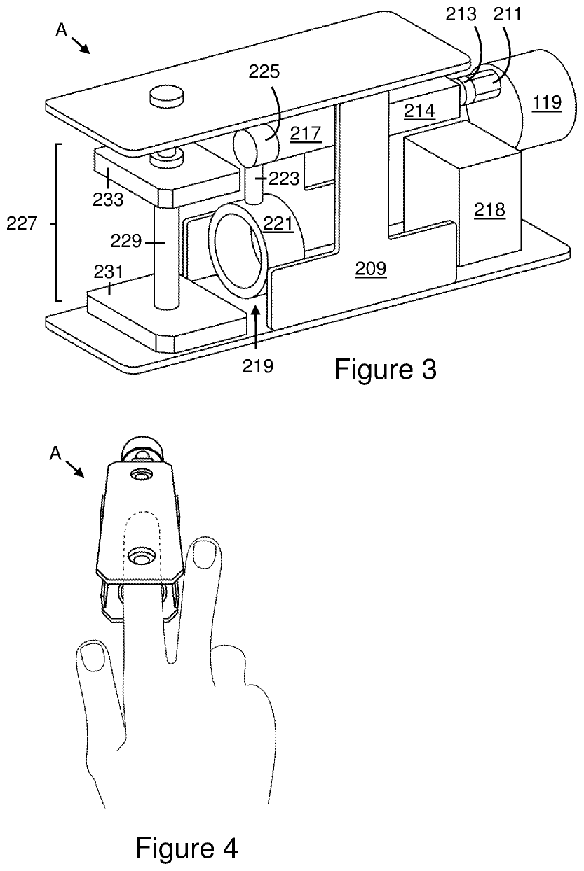

FIG. 3 illustrates features of an embodiment of the automatic distractor device (A). The reference numerals of FIGS. 1 and 2 are used to indicate the same or similar features.

FIG. 4 shows an embodiment of the automatic distractor device (A), viewed from a proximal dorsal side of a patient's hand. The device (A) is shown engaged with a patient's right forefinger, in a starting condition prior to performing DO on the finger.

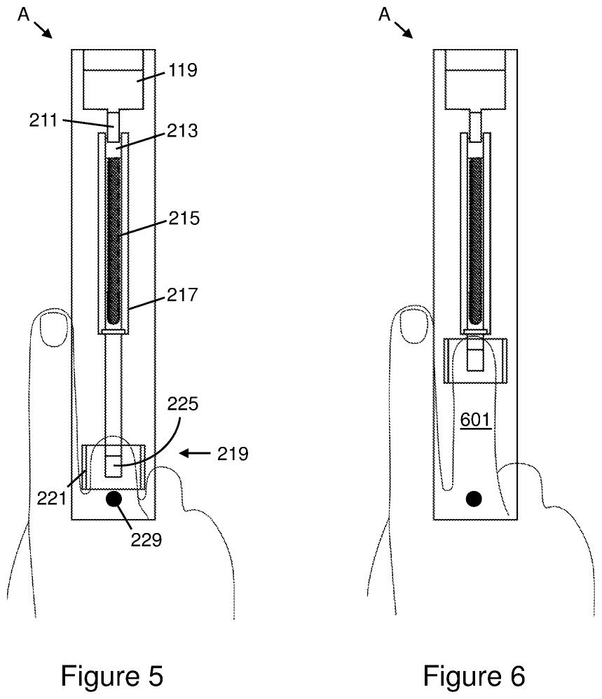

FIG. 5 shows the automatic distractor device (A) in a starting condition engaged with a patient's middle finger. The linear actuator (217) can be used to apply a distraction force between the distractor component (219) and the bone fixture component (227) which is fixed to a proximal BS of the finger by the screw (229).

FIG. 6 shows the same hand and automatic distractor device (A) as are shown in FIG. 6, but in this case after activation and operation of the system to lengthen the patient's middle finger. The linear actuator is shown nearing a destination position after elongation of a bone segment in the finger by osteogenesis. As can be seen by comparing FIGS. 5 and 6, the linear actuator (217) has now moved the distractor component (219) and a distal portion of the finger along a linear trajectory away from the bone fixture component (227), thereby generating bone tissue (601) by automatic distraction osteogenesis.

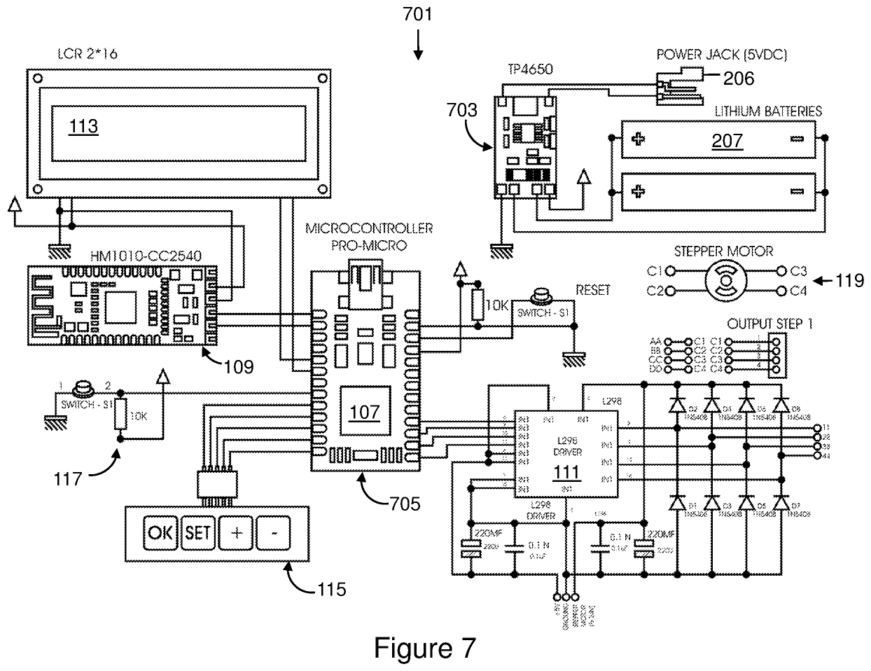

FIG. 7 is a schematic circuit diagram which illustrates an example of a control component (701) for an embodiment of the disclosed system. A TP4650 Lithium battery charger (703) and protection module is used to supply 5 VDC to the system, and to charge the Lithium-ion batteries (207) via the power jack (206).

A Pro-Micro™ development circuit board (705) with an ATMEGA32U4-AU™ microcontroller (107) is used. The microcontroller (107) has 12 digital input/output pins and a hardware serial connection, with a clock speed of 16 MHz. It will be appreciated that other suitable circuit boards and microcontrollers may be used. For example, in other embodiments the microcontroller may comprise a MAC (Machine Automation Controller).

A CC2540 Bluetooth® System-on-Chip (109) is included for wireless communication.

A 2 line by 16 character serial character liquid crystal display (LCD) (113) is connected to the controller (107), for displaying operating parameters and setting working factors before and during the bone reconstruction or generation process. A 4-key keypad (115) is provided to enable a user of the system to input data. The LCD (113) and keypad (115) can be removed from the device after the data has been input and the working factors or parameters have been set.

A mini hybrid stepper motor (119) and gearbox are included, along with a precise linear control system, multi-axis automatic controller or motor driver (111). A model 28BY J-48 motor is one non-limiting example of a suitable stepper motor that could be used. However, it will be appreciated that numerous other types and models of stepper motors may be suitable, and the use of these alternatives also falls within the scope of the present invention.

The motor driver (111) is configured to drive the stepper motor (119) and control the position of the linear actuator (217), and thus the movement of the BS.

The control component may be configured with an open-loop control system for controlling the distraction process. An example of a suitable set of control principles for the open-loop control system may be found in Hatefi et al. (2017). In other embodiments of the system, a closed-loop control system may be used instead, based on position feedback.

The control component of the system may be arranged for storing and executing computer program code. It may include subsystems or components interconnected via a communication infrastructure (for example, a communications bus, a network, etc.). The control component may include one or more processors and at least one memory component in the form of computer-readable media. The one or more processors may include one or more of: CPUs, graphical processing units (GPUs), microprocessors, field programmable gate arrays (FPGAs), application specific integrated circuits (ASICs) and the like. In some configurations, a number of processors may be provided and may be arranged to carry out calculations simultaneously. In some implementations various subsystems or components of the control component may be distributed over several physical locations (e.g., in a distributed, cluster or cloud-based computing configuration) and appropriate software units may be arranged to manage and/or process data on behalf of remote devices.

The present disclosure also provides a method for performing distraction osteogenesis on an anatomical extremity of a human or animal subject such as a hand. In this method, a proximal bone fixture component may be fixed to a proximal portion of a bone segment of the anatomical extremity. A movable distal distractor component may be utilised to engage a region of the anatomical extremity positioned distal of the bone portion engaged by the bone fixture component.

Drive means may be utilised to apply a distraction or separating force between the bone fixture component and the distractor component, and operation of the drive means may be controlled automatically using a control component.

### Example of Use

As a non-limiting example of use, the disclosed system could be used to perform ACDO during reconstruction of a finger by vascularized toe transfer. A finger portion formed from the grafted toe may thus be lengthened.

A distraction rate between about 0.5 mm/day and about 5 mm/day may, for example, be employed and the system may be set accordingly. A distraction rate in a narrower range, from about 3 mm/day to about 5 mm/day, may advantageously be employed.

Following the DO process, a surgery may be performed to remove the automatic distractor device (A) and stabilize the reconstructed bone in the desired position.

It will be appreciated that the potential uses of the disclosed system are not limited to reconstruction of bones. The system may also be used for lengthening of short or grafted fingers, for example.

The disclosed system may be suitable to provide an accurate, reliable, and stable continuous distraction force. It may provide key operational elements required for ACDO of hand bones, such as the rate and the rhythm of the distraction, the distraction vector, and the output distraction force generated by the automatic distractor device.

The embodiments of the system shown in the drawings are configured primarily for use on human hands. Therefore, their intended use is for lengthening, reconstructing or regenerating pre-existing or grafted hand bones. However, the scope of the invention may extend to embodiments of the system that are adapted for use with the bones of other anatomical extremities. For example, the system may be adapted to perform osteogenesis of feet bones, or even the bones of paws, talons, claws, or fins of animals. Modifications to the shapes, dimensions and configurations of the fixture component, distractor component, drive means, control component and other features may be required to adapt the system for use with such other types of anatomical extremities. The relevant structure of the targeted anatomical extremity, and the shapes, dimensions, and structural strength of the bones thereof, will need to be considered along with other relevant properties.

The presently disclosed system and method may have several advantages. To date there have been no solutions or devices available for reconstructing hand bones using automatic continuous distraction osteogenesis. Manual distractors have typically been used in the reconstruction of hand bones. Thus, previous studies reporting the successful reconstruction of acquired and congenital shortening of hand bones have relied upon manually performed DO technique requiring intermittent activation.

By contrast, the presently disclosed system may be used to provide a smooth, automatic, and continuous pulling or pushing force on a hand bone. The disclosed system may therefore extend the field to ACDO of hand bones. It may be useful to generate a controlled continuous force, to pull and move the BS during the activation phase of treatment.

Use of the disclosed system for reconstructing hand bones may be expected to produce high-quality regenerated bone tissue and enhanced bone healing in a shorter treatment time than traditional techniques relying on manual distraction osteogenesis.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

Finally, throughout the specification, unless the context requires otherwise:
“comprise” or variations such as “comprises” or “comprising” will be understood to imply the inclusion of a stated integer or group of integers but not the exclusion of any other integer or group of integers; and
the terms “finger” and “fingers” will be understood to include fingers and thumbs.

### REFERENCE

Hatefi, S., O. Ghahraei, and B. Bahraminejad, Design and Development of a Novel Multi-Axis Automatic Controller for Improving Accuracy in CNC Applications. Majlesi Journal of Electrical Engineering, 2017. 11(1).

1. A system for performing distraction osteogenesis of a bone segment of an anatomical extremity of a human or animal subject, said system comprising:
a proximal bone fixture component configured to engage a proximal portion of said bone segment;
a movable distractor component configured to be positionable distal of the proximal bone fixture component, and to engage a region of said anatomical extremity located distal of the bone portion engageable by the bone fixture component;
drive means configured to apply a distraction force between the bone fixture component and the movable distractor component; and
a control component arranged to control operation of said drive means automatically.

2. The system according to claim 1, wherein the anatomical extremity comprises a hand.

3. The system according to claim 1, an wherein the control component is configured to operate the drive means in a continuous manner.

4. The system according to claim 3, wherein the control component includes a user interface module for setting parameters of automatic operation and control of the drive means.

5. The system according to claim 4, wherein the user interface module is configured to be separable in a tool-free manner from the remainder of the control component.

6. The system according to claim 1, wherein the proximal bone fixture component and the movable distractor component interconnect via a transitional arrangement comprising force transfer means configured to apply the distraction force between the components.

7. The system according to claim 1, wherein the drive means is configured as a linear drive means connected to the movable distractor component and configured to move the distractor component along a linear vector away from the bone fixture component.

8. The system according to claim 1, wherein the movable distractor component comprises grip means for gripping the distal region of the anatomical extremity.

9. A method for performing distraction osteogenesis of a bone segment of an anatomical extremity of a human or animal subject, comprising the steps of
fixing a proximal bone fixture component to a proximal portion of the bone segment;
utilising a movable distractor component to engage a region of said anatomical extremity positioned distal of said proximal bone portion;
utilising a drive means to apply a distraction force between the bone fixture component and the movable distractor component; and
controlling operation of said drive means automatically using a control component.

10. The method according to claim 9, wherein the anatomical extremity comprises a hand.

11. The method according to claim 9, which includes operating the control component to drive the drive means in a continuous manner.

12. The method according to claim 9, wherein the control component includes a user interface module for setting parameters of automatic operation and control of the drive means, and wherein the method includes setting such parameters with the user interface module.

13. The method according to claim 12, wherein the user interface module is configured to be separable from the control component in a tool-free manner and the method includes separating the module from the remainder of the control component after said parameters have been set.

14. The method according to claim 9, wherein the proximal bone fixture component and the movable distractor component interconnect via a transitional arrangement comprising force transfer means configured to apply the distraction force between the components.

15. The method according to claim 9, which includes utilizing the drive means to move the distractor component along a linear vector away from the proximal bone fixture component.
